(19) United States
(12) Patent Application Publication
(10) Pub. No.: US 2009/0076596 A1
(43) Pub. Date: Mar. 19, 2009

(54) STENT HAVING A COATING

(75) Inventors: Nina Adden, Nuernberg (DE); Alexander Borck, Aurachtal (DE)

Correspondence Address: BRYAN CAVE POWELL GOLDSTEIN, ONE ATLANTIC CENTER FOURTEENTH FLOOR, 1201 WEST PEACHTREE STREET NW, ATLANTA, GA, 30309-3488, US

(73) Assignee: BIOTRONIK VI PATENT AG, Baar (CH)

(21) Appl. No.: 12/208,808

(22) Filed: Sep. 11, 2008

(30) Foreign Application Priority Data
Sep. 14, 2007 (DE) 10 2007 043 883.6

(51) Int. Cl. A61F 2/06

(52) U.S. Cl. 623/1.46

(57) ABSTRACT

A stent having a coating comprising either L-3,4-dihydroxyphenylalanine (L-DOPA) or a derivative of L-3,4-dihydroxyphenylalanine (L-DOPA), the foregoing obtained by either ionic or covalent bonding of a pharmaceutical active ingredient to either the amino or the acid function of L-3,4-dihydroxyphenylalanine (L-DOPA).

### PRIORITY CLAIM

This patent application claims priority to German Patent Application No. 10 2007 043 883.6, filed Sep. 14, 2007, the disclosure of which is incorporated herein by reference in its entirety.

### FIELD

The present disclosure relates to a stent having a coating.

### BACKGROUND

The implantation of stents has been established as one of the most effective therapeutic measures in the treatment of vascular illnesses. Stents provide a support function in the hollow organs of a patient. Stents of typical construction have filigree support structure made of metallic struts for this purpose. The stent is first provided in a compressed form for introduction into the body and is then expanded at the location of application. One of the main areas of application of such stents is permanently or temporarily expanding and keeping open vascular constrictions, in particular, constrictions (stenoses) of the coronary vessels. In addition, for example, aneurysm stents are also known which are used to support damaged vascular walls.

Stents have a peripheral wall of sufficient supporting force to keep the constricted vessel open to the desired degree and a tubular main body through which the blood flow continues to run unimpeded. The supporting peripheral wall is typically formed by a latticed support structure which allows the stent to be inserted in a compressed state having a small external diameter up to the constriction point of the particular vessel to be treated and to be expanded there with the aid of a balloon catheter, for example, enough that the vessel has the desired, enlarged internal diameter.

The stent has a main body made of an implant material. An implant material is a nonliving material which is used for an application in medicine and interacts with biological systems. The basic requirement for the use of a material as an implant material, which is in contact with the bodily environment when used as intended, is its biocompatibility. For purposes of the present disclosure, biocompatibility is the capability of a material to cause a suitable tissue reaction in a specific application. This includes an adaptation of the chemical, physical, biological, and morphological surface properties of an implant to the receiving tissue with the goal of a clinically desirable interaction. The biocompatibility of the implant material is also a function of the time sequence of the reaction of the biosystem in which the implant is implanted. Thus, relatively short-term irritations and inflammations occur which may result in tissue changes. Biological systems accordingly react in various ways as a function of the properties of the implant material. The implant materials may be divided into bioactive, bioinert, and degradable/resorbable materials according to the reaction of the biosystem.

Implant materials for stents comprise polymers, metallic materials, and ceramic materials (for example, as the coating). Biocompatible metals and metal alloys for permanent implants comprise rustproof steels (e.g., 316L), cobalt-based alloys (e.g., CoCrMo cast alloys, CoCrMo forged alloys, CoCrWNi forged alloys, and CoCrNiMo forged alloys), pure titanium and titanium alloys (e.g., cp titanium, TiAl₆V₄, or TiAl₆Nb₇), and gold alloys. In the field of biocorrodible implants, the use of magnesium or pure iron as well as biocorrodible base alloys of elements magnesium, iron, zinc, molybdenum, and tungsten is desirable.

A biological reaction to polymer, ceramic, or metallic elements is a function of the concentration, action time, and type of the supply. The presence of an implant material frequently results in inflammation reactions whose triggers may be mechanical irritations, chemical materials, or metabolic products among other things. The inflammation reaction is typically accompanied by the immigration of neutrophilic granulocytes and monocytes through the vascular walls, the immigration of lymphocyte effector cells with formation of specific antibodies against the inflammation stimulus, the activation of the complementary system with release of complementary factors which act as mediators, and finally the activation of blood coagulation. An immunological reaction is usually closely connected to the inflammation reaction and may result in sensitization and allergy formation. Known metallic allergens comprise, for example, nickel, chromium, and cobalt, which are also used as alloy components in many surgical implants. A significant problem of stent implantation in blood vessels is in-stent restenosis because of excessive neointimal growth which is caused by a strong proliferation of the arterial smooth muscle cells and a chronic inflammation reaction.

It is known that a higher degree of biocompatibility and thus an improvement of the restenosis rate may be achieved if implant materials are provided with coatings made of especially tissue-compatible materials. These materials are usually of an organic or synthetic polymer nature and are partially of natural origin. Further strategies for avoiding restenosis are concentrated on inhibiting proliferation by medication, e.g., treatment using cytostatics. The active ingredients may be provided on the implant surface in the form of a coating, for example.

In spite of the progress achieved, there is still a strong need to achieve a better integration of the stent in its biological surroundings and thus lower the restenosis rate.

### SUMMARY

The present disclosure describes several exemplary embodiments of the present invention.

One aspect of the present disclosure provides a stent having a coating, the coating comprising either L-3,4-dihydroxyphenylalanine (L-DOPA) or a derivative of L-3,4-dihydroxyphenylalanine (L-DOPA), wherein the derivative is obtained by ionic or covalent bonding of a pharmaceutical active ingredient to either the amino or the acid function of L-3,4-dihydroxyphenylalanine (L-DOPA).

The present disclosure is based on the finding that L-DOPA (IUPAC name: (2S)-2-amino-3-(3,4-dihydroxyphenyl)propanoic acid) and its derivatives have an especially high adhesive capability on common implant materials and thus particularly meet the specific requirements for stents whose surface is subjected to high mechanical forces when used as intended. L-DOPA is known to be a main component of the adhesive which mussels use to adhere to solid surfaces. The bonding predominantly occurs via the two OH groups and, therefore, a combination of L-DOPA and its derivatives with main bodies of stents which have a polar surface results, in particular, in coatings having a very high adhesive capability. In contrast to common monolayers, such as silane layers, L-DOPA may not only bond to metal but rather L-DOPA may also bond solidly to all services relatively nonspecifically.

L-DOPA is a non-proteinogenic α-amino acid which is formed in the body from tyrosine with the aid of the enzyme tyrosine hydroxylase. The compound is used as a pharmaceutical under the name Levodopa. When used as intended as a coating material for stents, a high biocompatibility is, therefore, to be expected.

L-DOPA has a 2-aminopropanoic acid residue via whose two functionalities pharmaceutically active compounds may be bonded. If the active ingredients used are acids, bases, or salts, the bond may be based on ionic interaction with the two functionalities of the L-DOPA. Alternatively, a covalent bond of the active ingredient via the acid or amino function is possible. The covalent bond may be composed in such a way that cleavage and thus controlled release of the active ingredient in the body again are possible. The active ingredient is accordingly introduced with the implantation in the meaning of a prodrug and is first converted into the active form in the organism. However, it is also conceivable that the covalent bond remains in existence in vivo and the active ingredient is immobilized on the implant surface. L-DOPA and its derivatives are suitable for a plurality of surfaces in addition to the metallic surfaces. The advantage of the method is the nonspecific bonding capability.

The production of the stent having a coating which contains such a derivative of L-DOPA or comprises such a derivative of L-DOPA may preferably be performed via a stent which has a coating which comprises or contains L-DOPA. In other words, a stent coated with L-DOPA is first produced. Subsequently, the active ingredient is ionically or covalently bonded via the L-DOPA immobilized on the implant surface.

For example, aptamers, RGD sequences, antibodies, such as CD 133, anticoagulants, proliferation inhibitors, inflammation inhibitors, calcium channel blockers, and endothelin receptor antagonists and others may be used as the active ingredients. Preferred active ingredients particularly comprise cRGD, dODNs, Bosentan, Antisence, Sirolimus, and its derivatives (such as Biolimus and Everolimus) as well as Pimecrolimus.

Aptamers are short single-strand DNA or RNA oligonucleotides (25-70 bases) which may bond a specific molecule via their 3-D structure. Aptamers bond to proteins, such as growth factors and bacterial toxins, low-molecular-weight materials, such as amino acids and antibiotics, and also to virus particles. Aptamers are distinguished by high specificity and affinity, high chemical stability, low immunogenicity, and the capability for targeted influencing of protein-protein interactions. Aptamers, and particularly spiegelmers, are protected from DNAses (enzymes which degrade DNA) by their conformation and have a sufficiently high half-life in the body.

For purposes of the present disclosure, an RGD sequence is an amino acid sequence made of the three amino acids arginine, glycine, and asparagine acid, Arg-Gly-Asp in short, or RGD in the single-letter code. The RGD sequence occurs, in particular, in proteins of the extracellular matrix (connective tissue), for example, in fibronectin and vitronectin. Cells may bond to the RGD sequence with the aid of specific cell surface receptors, the integrins. RGD-mediated cell adhesion is used for mechanical anchoring of cells. Implants may be coated with the RGD sequence for better integration in the body. The RGD sequence is present in the various matrix proteins in differing conformation which is partially recognized specifically by the integrin subtypes (for example, cyclic RGD peptides, such as cilengitides). cRGD have a conformation which allows them to couple selectively to integrins of specific cells. It is thus possible to differentiate between SMC and EC cells. Adhesion may be forced or prevented by the bonding to the bonding domains of the integrins.

Antibodies (immunoglobulins) are proteins from the class of globulins which are formed in vertebrates as a reaction to specific intruding foreign materials, referred to as antigens. Antibodies are used to defend from these foreign materials. A specific antigen typically only induces the formation of a specific antibody matching therewith, which is specifically bound only to this foreign material. The use of antibodies CD 133 and CD 34 is preferred.

The stent preferably has a metallic main body. The metallic main body particularly comprises magnesium, a biocorrodible magnesium alloy, pure iron, a biocorrodible iron alloy, a biocorrodible tungsten alloy, a biocorrodible zinc alloy, or a biocorrodible molybdenum alloy.

For purposes of the present disclosure, alloys and elements in which a degradation/conversion occurs in a physiological environment, so that the part of the implant comprising the material is entirely or at least predominantly no longer present, are referred to as biocorrodible.

For purposes of the present disclosure, a magnesium alloy, iron alloy, zinc alloy, molybdenum alloy, or tungsten alloy is a metallic structure whose main component is magnesium, iron, zinc, molybdenum, or tungsten. The main component is the alloy component whose weight proportion in the alloy is the highest. A proportion of the main component is preferably more than 50 wt.-%, in particular, more than 70 wt.-%. The alloy is to be selected in its composition in such a way that the alloy is biocorrodible. Artificial plasma, as has been previously described according to EN ISO 10993-15:2000 for biocorrosion assays (composition NaCl 6.8 g/l, CaCl₂ 0.2 g/l, KCl 0.4 g/l, MgSO₄ 0.1 g/l, NaHCO₃ 2.2 g/l, Na₂HPO₄ 0.126 g/l, NaH₂PO₄ 0.026 g/l), is used as a testing medium for testing the corrosion behavior of an alloy. For this purpose, a sample of the alloy to be assayed is stored in a closed sample container with a defined quantity of the testing medium at 37° C. At time intervals, tailored to the corrosion behavior to be expected, of a few hours up to multiple months, the sample is removed and examined for corrosion traces in a way known in the art. The artificial plasma according to EN ISO 10993-15:2000 corresponds to a medium similar to blood and thus simulates a reproducible physiological environment.

### DETAILED DESCRIPTION

The present disclosure is explained in greater detail hereafter on the basis of an exemplary embodiment.

A stent made of the biocorrodible magnesium alloy WE43 (according to ASTM) is degreased and dried. The coating is performed as follows.

The stent is immersed at a pH value 8 through 10 in PBS buffer for 10 minutes up to one hour in a 2% L-DOPA solution. Subsequently, the stent is removed from the solution, washed using ultrapure water, and dried.

The functionalized stent is placed for 3 to 5 hours in N,N′-carbonyldiimidazole (CDI). For purposes of the present disclosure, the CDI is dissolved in dry dioxane. A starting solution of 2.5 g/50 ml CDI in dioxane is suitable for this purpose and may be stored for several days (2, dry). The stent is moved easily at room temperature.

After the activation, the stents are removed and washed using dry dioxane.

For the coupling of antibodies, the activated stents are immersed in the antibody solution and coupled at 4° C. overnight (at least 12 hours). The reaction occurs most suitably in 125 mM sodium borate having 0.066% SDS at a pH value of 10.

The solution is then reusable and/or multiple surfaces may be treated using this solution.

The stents are washed three times with 5 mL of the borax buffer (above) after the coupling and then are washed three times with water. The antibodies still analyzable after these washing steps are covalently bound.

What is claimed is:

1. A stent having a coating, the coating comprising: either L-3,4-dihydroxyphenylalanine (L-DOPA) or a derivative of L-3,4-dihydroxyphenylalanine (L-DOPA), the foregoing obtained by either ionic or covalent bonding of a pharmaceutical active ingredient to either the amino or the acid function of L-3,4-dihydroxyphenylalanine (L-DOPA).

2. The stent of claim 1, wherein the stent has a metallic main body which carries the coating.

3. The stent of claim 1, wherein the metallic main body of the stent comprises a material selected from the group consisting of magnesium, a biocorrodible magnesium alloy, pure iron, a biocorrodible iron alloy, a biocorrodible tungsten alloy, a biocorrodible zinc alloy, and a biocorrodible molybdenum alloy.
